(19) United States
(12) Patent Application Publication
(10) Pub. No.: US 2005/0009744 A1
(43) Pub. Date: Jan. 13, 2005

(54) Treatment of congestive heart failure

(76) Inventors: Olle Isaksson, Goteborg (SE); Jorgen Isgaard, Goteborg (SE); Gudmundur Johannsson, Vastra Frolunda (SE)

Correspondence Address: BURNS DOANE SWECKER & MATHIS L L P, POST OFFICE BOX 1404, ALEXANDRIA, VA, 22313-1404, US

(21) Appl. No.: 10/494,328

(22) PCT Filed: Nov. 8, 2002
(86) PCT No.: PCT/SE02/02048

(30) Foreign Application Priority Data
Nov. 8, 2001 (SE) 0103749-8

(51) Int. Cl. A61K 38/28; A61K 38/30

(52) U.S. Cl. 514/12

(57) ABSTRACT

The use of at least one first substance related to the growth hormone (GH) axis, and of at least one second substance, wherein the second substance upon administration to a patient leads to increased beta adrenergic receptor blockade, for the production of a pharmaceutical composition for the treatment of congestive heart failure (CHF) is disclosed. The use of the at least one first or second substance, respectively, for the production of a pharmaceutical composition for the treatment of congestive heart failure (CHF), intended for administration in combination with a pharmaceutical composition comprising the at least one second or first substance, respectively, is also disclosed. Also a method for treatment of congestive heart failure (CHF), wherein a pharmaceutically active amount of the at least one first substance is administered to a patient in combination with the administration to a patient of a pharmaceutically active amount of the at least one second substance is disclosed.

### BACKGROUND OF THE INVENTION

Despite significant advances in therapy, congestive heart failure is still the leading cause of mortality in the Western world (Gheorghiade & Bonow Circulation 1998; 97: 282-289). Ischemic heart disease is the principal cause of congestive heart failure (CHF) and almost 50% of patients surviving myocardial infarction (MI) eventually develop heart failure (Gheorghiade & Bonow Circulation 1998; 97: 282-289). Despite recent advances in the treatment, severe CHF remains a syndrome associated with high mortality, therefore, the search for new agents to improve both patient symptoms and survival continues. Large acute myocardial infarction can initiate complex changes in the geometrical, structural, and biochemical architecture of both infarcted and non-infarcted regions of ventricular myocardium, which can profoundly affect left ventricular (LV) function and prognosis. Since the Göteborg metoprolol trial (Hjalmarson et al, Lancet 1981; 2: 823-827, Hjalmarson et al, Circulation 1983; 67: 126-32), several others have confirmed the beneficial effects of beta-blockers in acute MI. These range from a 13% reduction in mortality in the first 24 h after early intravenous beta-blockade to substantial reduction in mortality and morbidity after chronic therapy in post myocardial infarction patients and patient with CHF (Eichhorn & Bristow Circulation 1996; 94: 2285-2296).

Recent experimental data have demonstrated beneficial effects of growth hormone (GH) in the treatment of postinfarct remodeling and heart failure (Cittadini et al, J Am Coll Cardiol 1997; 29: 1109-1116, Duerr et al, Circulation 1996; 93: 2188-2196, Isgaard et al, Eur J Clin Invest 1997; 27: 517-525, Yang et al, 1995; 92: 262-267). Moreover, early start of GH treatment after MI has been shown to prevent pathologic remodeling and to improve LV function (Cittadini et al., J Am Coll Cardiol 1997; 29: 1109-1116). Decreased peripheral resistance (Caidahl et al, Clin Endocrinol 1994; 40: 393-400) induction of a “physiologic hypertrophy” (Cittadini et al, J Am Coll Cardiol 1997; 29: 1109-1116) improvement of intra-cellular Ca²⁺ handling (Xu & Best Proc Natl Acad Sci 1990; 87: 4655-4659) and increased force of contraction without increased energy expenditure (Timsit et al, Acta Paediatr Suppl 1992; 383: 32-34), are some of the proposed mechanisms of GH action on the cardiovascular system. Small open clinical studies have demonstrated beneficial effects of GH on hemodynamics and clinical function in patients with congestive heart failure (Fazio et al, N Engl J Med 1996; 334-809-814, Genth-Zotz et al, Circulation 1999; 99: 18-21) although these findings have so far not been confirmed in placebo controlled trials (Osterziel et al, Lancet 1998; 351: 1233-1237, Isgaard et al, Eur Heart J 1998; 19: 1704-1711).

r-hGH, alone or in combination with one of the substances presently used for the treatment of chronical congestive heart failure, has been suggested (Dreifuss, Z Kardiol 1998; 87: 425-435). Among the presently used substances, the betablocker carvedilol is mentioned.

However, carvedilol is in many ways quite different from other beta blockers. Firstly, carvedilol is a multiple-action neurohormonal antagonist, i.e. a non-selective drug with combined alpha and beta blocking properties. Other beta blockers, such as e.g. sotalol, atenolol, and metoprolol, are highly selective beta antagonists (see e.g. Khandoudi et al, J Cardiovasc Pharmacol Sep. 1998; 32(3): 443-51).

Secondly, carvedilol has a great antioxidant activity, which could account for its favourable effects on heart disease. Carvedilol has been shown to possess far greater antioxidant activity than e.g. metoprolol, which is essentially inactive as an antioxidant (Lysko et al, J Cardiovasc Pharmacol 2000; 36(2): 277-81).

Until now the therapeutic effects of the combination of GH and beta blockade in patients with congestive heart failure have not been properly evaluated.

The therapy of the acute MI and post MI remodeling is complex and consists of a combination of three or more pharmaceutical agents including nitrates, ACE-inhibitors and beta-blockers. Moreover, combination of beta-blockers and angiotensin-converting enzyme inhibitors appears to offer additive benefits (Cohn et al, J Am Coll Cardiol 2000; 35: 569-582). Theoretically, addition of GH in the early phase after MI could have additive positive effects. However, an early report from Castagnino et al (Castagnino et al, Jpn Heart J 1990; 31: 845-855), showed a negative impact of the combined treatment of GH and betablockade with increased prevalence of LV aneurysm after MI in an experimental model.

### SUMMARY OF THE INVENTION

The inventors have found that a combined treatment of growth hormone (GH) and beta adrenergic receptor blockade markedly improved exercise capacity and cardiac output in patients with congestive heart failure (CHF).

The invention thus relates to the use of substances related to the growth hormone (GH) axis in combination with substances, which upon administration to a patient leads to increased beta adrenergic receptor blockade.

More precisely, the invention relates to the use of at least one first substance related to the growth hormone (GH) axis, and of at least one second substance, wherein said second substance upon administration to a patient leads to increased beta adrenergic receptor blockade, for the production of a pharmaceutical composition for the treatment of congestive heart failure (CHF).

The invention also relates to the use of at least one first substance related to the growth hormone (GH) axis for the production of a pharmaceutical composition for the treatment of congestive heart failure (CHF), intended for administration in combination with a pharmaceutical composition comprising at least one second substance, wherein said second substance upon administration to a patient leads to increased beta adrenergic receptor blockade.

Further, the invention relates to the use of at least one second substance, which upon administration to a patient leads to increased beta adrenergic receptor blockade, for the production of a pharmaceutical composition for the treatment of congestive heart failure (CHF), intended for administration in combination with a pharmaceutical composition comprising at least one first substance related to the growth hormone (GH) axis.

The invention also relates to a method for treatment of congestive heart failure (CHF), wherein a pharmaceutically active amount of at least one first substance related to the growth hormone (GH) axis is administered to a patient in combination with the administration to a patient of a pharmaceutically active amount of at least one second substance, wherein said second substance upon administration to a patient leads to increased beta adrenergic receptor blockade.

Other features, as well as advantages of the present invention will be evident from the following description and the appended claims.

### DETAILED DESCRIPTION OF THE INVENTION

As stated above, the invention relates i.a. to the use of substances related to the growth hormone axis. The expression “substance related to the growth hormone axis” relates to all substances related to, linked to or involved in the sequence of successive activation reactions wherein GH is involved, comprising GH itself, hormones from the hypothalamus affecting GH and hormones or growth factors affected by GH. Preferred examples of such substances are growth hormone (GH), growth hormone secretagogues (GHSs), or insulin like growth factor I (IGF-I). More specifically, substances increasing the levels of growth hormone are preferred. Especially preferred is GH or an analogue thereof.

When growth hormone (GH) is used according to the invention it is preferably human growth hormone. It is possible to use both naturally derived GH and synthetically produced GH. GH is also called somatotropin or somatotropic hormone.

A growth hormone secretagogue (GHS) is a substance that stimulates secretion of GH. According to the invention it is possible to use both naturally derived GHSs and synthetically produced GHSs. One exemple of a naturally derived GHS is the endogen substance ghrelin. Furthermore, the GHS used may be either a peptidic or a non-peptidic substance. One example of a peptidic GHS suitable for use according to the present invention is hexarelin, which is a substance that leads to increased secretion of GH.

The insulin like growth factor I (IGF-I) used according to the invention is preferably human IGF-I. It is possible to use both naturally derived IGF-I and synthetically produced IGF-I.

It is also possible to use functionally equivalent analogues of GH, GHSs, or IGF-I. The expression “functionally equivalent analogue” used herein relates to any substance that is structurally similar to GH, GHSs, or IGF-I and has essentially the same pharmacological and/or therapeutical effects.

Furthermore, it is possible to use a combination of two or more of the substances related to the GH axis. It is, however, not necessary to use more than one of these substances. Thus, in the most preferred embodiment GH, or an analogue thereof, is used.

The invention also relates to the use of substances that upon administration to a patient leads to increased beta adrenergic receptor blockade. The expression “increased beta adrenergic receptor blockade” relates to all substances that upon administration result in a decrease of the atecholamine effects on the heart. Examples of such substances for use ccording to the invention are sotalol, atenolol, metoprolol, propranol, isoprolol, and labetalol. It is also possible to use functionally equivalent analogues of these substances. The expression “functionally equivalent analogue” is defined above.

Furthermore, it is possible to use a combination of two or more of the substances that upon administration to a patient leads to increased beta adrenergic receptor blockade. It is, however, not necessary to use more than one of these substances. Preferably, metoprolol, sotalol or atenolol is used.

The substance related to the GH axis and the substance that upon administration to a patient leads to increased beta adrenergic blockade may be comprised in separate pharmaceutical compositions, intended to be used together.

In the method according to the invention, the substance related to the GH axis and the substance increasing the beta adrenergic receptor blockade are administrated in combination with each other, either consecutively or simultaneously. The consecutive administration can be done either by first administrating the substance related to the GH axis, or a pharmaceutical composition comprising it, and than the substance increasing the beta adrenergic receptor blockade, or a pharmaceutical composition comprising it, or by first administrating the substance increasing the beta adrenergic receptor blockade, or a pharmaceutical composition comprising it, and than the substance related to the GH axis, or a pharmaceutical composition comprising it.

The pharmacological composition produced according to the invention and the method according to the invention are suitable for treatment of congestive heart failure (CHF) of idiopathic, ischemic or other causes.

The term “patient”, as it is used herein, relates to any human or non-human mammal in need of treatment according to the invention.

The term “treatment” used herein, relates to both treatment in order to cure, or alleviate a disease or a condition, and to treatment in order to prevent the development of a disease or a condition. The treatment may be either performed in an acute or in a chronic way.

In the method according to the present invention, a “pharmaceutically active amount” of the substance is used. This expression relates to a dose of the substance that will lead to the desired pharmacological and/or therapeutic effect. The desired pharmacological effect is, as stated above, to cure or alleviate congestive heart failure (CHF).

The pharmaceutical composition according to the invention may also comprise other substances, such as an inert vehicle, or pharmaceutical acceptable adjuvants, carriers, preservatives, etc, which are well known to persons skilled in the art, provided that the curative or alleviating effect on CHF is not severely impaired.

Furthermore, it is possible to combine the treatment according to the invention with other treatments of CHF.

The invention will be further illustrated in the example below, which in no way is intended to limit the scope of the invention.

### BRIEF DESCRIPTION OF THE DRAWINGS

FIG. 1 is a diagram illustrating the cardiac output in two patients with CHF at baseline (white bars) and after three months of treatment with growth hormone and beta adrenergic receptor blockade (filled bars)

FIG. 2 is a diagram illustrating the excercise capacity in two patients with CHF at baseline (white bars) and after three months of treatment with growth hormone and beta adrenergic receptor blockade (filled bars)

### EXAMPLE

Patients

Two male patients with congestive heart failure who had been stable on treatment with beta adrenergic receptor blockade for at least 6 months were treated with recombinant human growth hormone manufactured by Pharmacia (Sweden) and marketed under the trademark Genotropin on top of continued treatment with beta adrenergic receptor blockade. Administration of growth hormone was achieved by daily subcutaneous injections.

Patient 1 was a male 69 years old at the start of treatment with GH. He suffered from congestive heart failure due to cardiac ischemia. He had been stable on tretament with beta adrenergic receptor blockade (sotalol, 80 mg daily taken orally) for >6 months) when he started treatment with recombinant human growth hormone in a daily dose of 3 IU (1 mg) for 3 months. He was in function class 1 according to NYHA classification at the start of GH treatment.

Patient 2 was a male 73 years old at the start of treatment with GH. He suffered from congestive heart failure due to cardiac ischemia and had had a previous myocardial infarction. He had been stable on treatment with beta adrenergic receptor blockade (atenolol 100 mg daily, taken orally) for >6 months) when he started treatment with recombinant human growth hormone in a daily dose of 3 IU (1 mg) for 3 months. He was in function class 2 according to NYHA classification at the start of GH treatment.

NYHA Function Class

Functional classification was done according to New York Heart Association functional classes I-IV (Criteria Committe, New York Heart Association, Inc.: Diseases of the heart and blood vessels. Nomenclature and criteria for diagnosis. 6th ed. Boston, Little, Brown and Co, 1964, p 114).

Doppler-Echocardiography

Doppler-echocardiography was performed using an Acuson-128 computed sonograph equipped with a 2 or 3.5 Mhz transducer. Two-dimensional echocardiography was performed in standard parasternal and apical projections to evaluate valvular abnormalities and rule out regional wall motion disturbances. M-mode echocardiography was used for the evaluation of left atrial end-systolic dimension, left ventricular end-diastolic and end-systolic dimensions, and left ventricular walls at end-diastole and end-systole, respectively. Measurements were made according to the recommendations of the American Society of Echocardiography (Sahn et al, Circulation 1978; 58: 1072-1083), end-diastole defined by the electrocardiographic Q-wave and end-systole as minimum left ventricular dimension or, for left atrial measurement, as aortic valve closure. Left ventricular fractional shortening was calculated as: (end-diastolic−end-systolic)/end-diastolic dimensions. End-systolic wall stress was calculated as earlier described (Reichek et al Circulation 1982; 65: 99-108). Stroke volume was measured by Doppler-echocardiography as left ventricular outflow tract (LVOT) area x velocity time integral of the Doppler flow. LVOT area was calculated from the long-axis diameter in parasternal view. Cardiac output was obtained by multiplying stroke volume by heart rate. Measurements were made at baseline and after 3 months of GH treatment.

Exercise Capacity

Maximal exercise capacity was determined by a symptom limited maximal sitting bicycle exercise test at baseline, and after three months of GH treatment. The starting load was 30 W and there was a 10 W increment per minute. Pulse rate, blood pressure, symptoms, arrhythmias and ST deflection were registered at each level. The given value of maximal exercise capacity represents the work load maintained for at least 45 sec.

Results

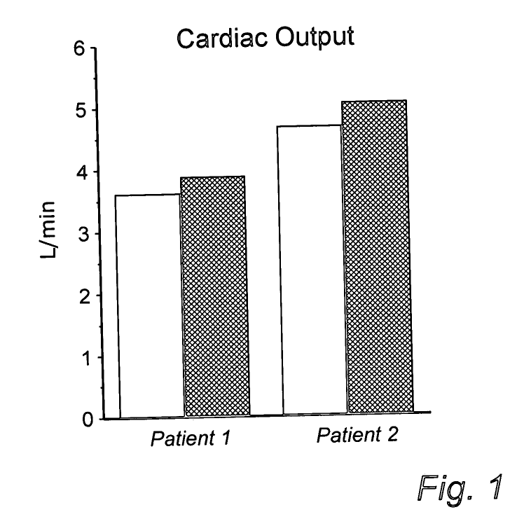

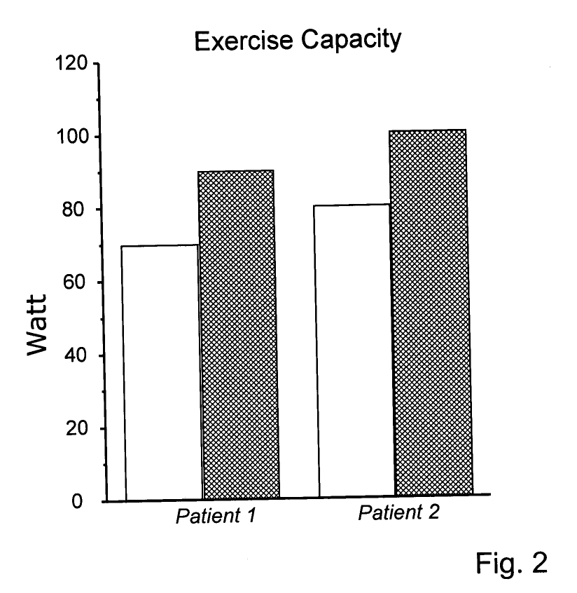

As can be seen in FIGS. 1 and 2, the cardiac output, as well as the exercise capacity, was improved in the CHF-patients after three months of treatment with growth hormone and beta adrenergic receptor blockade. These findings demonstrate that this treatment improves cardiac finction and increases physical performance in severely ill patients with poor prognosis.

1. (Cancelled).

2. A pharmaceutical composition for the treatment of congestive heart failure (CHF) comprising a pharmaceutically active amount of at least one first substance related to the growth hormone (GH) axis, selected from the group consisting of growth hormone (GH), growth hormone secretagogues (GHS), and insulin like growth factor I (IGF-I), or functionally equivalent analogues thereof, in combination with a pharmaceutically active amount of at least one second substance, selected from the group consisting of sotalol, atenolol, metoprolol, propranol, bisoprolol, and labetalol, or functionally equivalent analogues thereof, wherein said second substance upon administration to a patient leads to increased beta adrenergic receptor blockade.

3. (Canceled).

4. (Canceled).

5. A pharmaceutical composition according to claim 2, wherein said growth hormone secretagogue (GHS) is ghrelin or a functionally equivalent analogue thereof.

6. A pharmaceutical composition according to claim 2, wherein said growth hormone secretagogue (GHS) is a non-peptidic substance.

7. A pharmaceutical composition according to claim 2, wherein said growth hormone secretagogue (GHS) is a peptidic substance.

8. A pharmaceutical composition according to claim 7, wherein said peptidic substance is hexarelin or a functionally equivalent analogue thereof.

9. A method for treatment of congestive heart failure (CHF), wherein a pharmaceutically active amount of at least one first substance related to the growth hormone (GH) axis is administered to a patient in combination with the administration to a patient of a pharmaceutically active amount of at least one second substance, wherein said second substance upon administration to a patient leads to increased beta adrenergic receptor blockade.

10. A method according to claim 9, wherein said first substance upon administration to a patient leads to an increased level of growth hormone (GH).

11. A method according to claim 10, wherein said first substance is growth hormone (GH) or a functionally equivalent analogue thereof.

12. A method according to claim 10, wherein said first substance is a growth hormone secretagogue (GHS) or a functionally equivalent analogue thereof.

13. A method according to claim 12, wherein said growth hormone secretagogue (GHS) is ghrelin or a functionally equivalent analogue thereof.

14. A method according to claim 12, wherein said growth hormone secretagogue (GHS) is a non-peptidic substance.

15. A method according to claim 12, wherein said growth hormone secretagogue (GHS) is a peptidic substance.

16. A method according to claim 15, wherein said peptidic substance is hexarelin or a functionally equivalent analogue thereof.

17. A method according to claim 9, wherein said first substance is insulin like growth factor I (IGF-I) or a functionally equivalent analogue thereof.

18. A method according to claim 9, wherein said second substance is sotalol or a functionally equivalent analogue thereof.

19. A method according to claim 9, wherein said second substance is atenolol or a functionally equivalent analogue thereof.

20. A method according to claim 9, wherein said second substance is metoprolol or a functionally equivalent analogue thereof.

21. A method according to claim 9, wherein said second substance is propranol or a functionally equivalent analogue thereof.

22. A method according to claim 9, wherein said second substance is bisoprolol or a functionally equivalent analogue thereof.

23. A method according to claim 9, wherein said second substance is labetalol or a functionally equivalent analogue thereof.

24. A method according to claim 9, wherein said first substance and said second substance are consecutively administered.

25. A method according to claim 9, wherein said first substance and said second substance are simultaneously administered.
